(19) United States
(12) Patent Application Publication
(10) Pub. No.: US 2004/0199154 A1
(43) Pub. Date: Oct. 7, 2004

(54) Device for tissue ablation

(75) Inventors: Daniel Nahon, Ottawa (CA); Sean Carroll, Beaconsfield (CA); Michael Urick, Beaconsfield (CA); Marwan Abboud, Pierrefonds (CA)

Correspondence Address: John Christopher, Christopher & Weisberg, P.A., Suite 2040, 200 East Las Olas Boulevard, Fort Lauderdale, FL, 33301, US

(73) Assignee: CryoCath Technologies Inc.

(21) Appl. No.: 10/405,202

(22) Filed: Apr. 2, 2003

(51) Int. Cl. A61B 18/18

(52) U.S. Cl. 606/21; 606/41

(57) ABSTRACT

The present invention provides a medical device for guiding an ablation tool onto the surface of tissue. As described herein, the invention includes a device which can be shaped to reach around an organ, bone or tissue structure, and have an optimal configuration for positioning and or orienting the active or distal region of the device based upon the particular anatomy of a patient and the location of the treatment site.

### CROSS-REFERENCE TO RELATED APPLICATION

n/a

### STATEMENT REGARDING FEDERALLY SPONSORED RESEARCH OR DEVELOPMENT

n/a

### FIELD OF THE INVENTION

The invention relates to a medical device, and more particularly to a surgical instrument used for tissue ablation.

### BACKGROUND OF THE INVENTION

It is well documented that atrial fibrillation (AF), either alone or as a consequence of other cardiac disease, continues to persist as the most common type of cardiac arrhythmias. In the United States, AF currently affects an estimated two million people, with approximately 160,000 new cases being diagnosed each year. The cost of treatment for AF alone is estimated to be in excess of $400 million worldwide each year.

Although pharmacological treatment is available for AF, the treatment is far from perfect. For example, certain antiarrhythmic drugs, like quinidine and procainamide, can reduce both the incidence and the duration of AF episodes. Yet, theses drugs often fail to maintain sinus rhythm in the patient. Cardioactive drugs, like digitalis, Beta blockers, and calcium channel blockers, can also be given to control AF by restoring the hearts natural rhythm and limiting the natural clotting mechanism of the blood. However, antiarrhythmic drug therapy often becomes less effective over time. In addition, antiarrhythmic drug can have severe side effects, including pulmonary fibrosis and impaired liver function.

Another therapy for AF is surgery. In a technique known as the “Maze” procedure, a surgeon makes several slices through the wall of the atrium with a scalpel and then sews the cuts back together, creating a scar pattern. The scars isolate and contain the chaotic electrical impulses to control and channel the electrical signals. The Maze procedure is expensive, complicated to perform, and associated with long hospital stays and high morbidity.

An alternative to open heart or open chest surgery is a minimally invasive treatment in which ablation devices are used to form scars in various locations in the atrial tissue. Ablation devices that apply heat or cold to body tissue are known. Typically, these devices have an elongate, highly-flexible shaft with a steerable distal end for negotiating a path through the body of a patient, as well as having a rigid shaft for use in more invasive procedures where a more local opening or direct access to a treatment site is available or created.

While rigid shafts may be useful in some applications, they have certain limitations as well. For example, without a preset shape especially adapted for reaching a particular location in the body of a patient, the rigid nature of the shaft limits the area of tissue that can be reached and treated. Even where a relatively large incision is provided, tissue areas that are not at least somewhat directly accessible cannot be reached.

Although a rigid shaft can be provided with a predetermined shape, one must select a device with a rigid shaft that has the most appropriate shape for positioning the working portion of the device in contact with the treatment site in view of the particular anatomical pathway to be followed in the patient. It will be appreciated that a large inventory of devices having rigid shafts may be required to accommodate the various treatment sites and patient anatomies. As an example, U.S. Pat. No. 6,161,543 to Cox el al. describes a variety of rigid probe shapes. Further, for a patient having a relatively uncommon anatomic configuration and/or a difficult to reach treatment site, all rigid devices of an existing set may have less than optimal shapes for positioning. This may impair the prospects of successfully carrying out the treatment procedure. For an ablation device which must bear against tissue at the remote region to create lesions, the contour followed by the device in reaching the target site will in general further restrict the direction and magnitude of the movement and forces which may be applied or exerted on the working portion of the device to effect tissue contact and treatment.

Still other ablation devices have a steerable flexible shaft inventions, for example U.S. patent application Publication No. 20020087151 which describe flexible shaft guidance systems. While a steerable flexible shaft facilitates positioning of the catheter around an organ, the flexible shaft does not retain sufficient rigidity to aid in applying the force necessary to obtain good contact along the length of the device.

It would, therefore, be desirable to provide a malleable guiding device and ablation tool that, while having sufficient rigidity to facilitate guiding the device to a selected location within the body of a patient, is also better adapted to reach or treat the particular targeted anatomy of the patient.

It would also be desirable to provide a device having a working portion with sufficient controlled flexibility to conform to curved or irregular tissue surfaces, yet be resistant to kinking, folding or pinching. In addition the ablation tool should have sufficient strength to safely contain high-pressure working cryogenic fluids or other hardware necessary to deliver other ablative energies such as, but not limited to, ultrasound, radio frequency, laser, chemical, or microwave or a combination of thereof. For example, RF energy being delivered simultaneously with cryogenic fluids.

### SUMMARY OF THE INVENTION

The present invention provides a medical device and ablation tool for guiding the ablation tool onto the surface of tissue. As described herein, the invention includes a substantially tubular guiding device which can be shaped to reach around an organ, bone or tissue structure, and have an optimal configuration for positioning and or orienting an active or distal region of the device based upon the particular anatomy of a patient and the location of the treatment site. The guide can also be used to reach into an organ cavity, such as a heart chamber, bone cavity, or uterine cavity. In this instance the guide can serve the dual purpose of guiding the ablation tool and holding adjacent tissue away from the ablation tool. This can be particularly useful when performing ablation in large hypertrophied hearts. The ablation tool can be a probe or catheter and the two items are used interchangeably in this document.

The medical device contains a malleable or flexible guide which is shapeable with the application of moderate pressure. The shapeable feature allows the operator to bend the guide to a shape or contour with moderate pressure, dependent on the defined shape of the ablation. The guide should also be sufficiently rigid such that the surgeon can place the guide in pressure contact with the tissue treatment site without inducing further deformation in the shape of the guide.

Additionally, the guide is configured for receiving a flexible ablation tool, where at least a portion of the ablation tool is exposed to the tissue to be treated at the guide's distal portion. This configuration partially circumferences the ablating portion of the ablation tool, such that the guide acts as a shield preventing undesirable damage to tissue adjacent to the non-contact portion of the ablation tool.

In an example of use, the ablation tool is configured for cryogenic ablation and connected to a cryogenic fluid control system, which can include a controller operable connected to a cryogenic fluid supply. Based on the patient's anatomy and treatment site, the guide is shaped to achieve an optimal configuration for reaching and orienting the ablation segment in physical contact with the target tissue for ablating a line or contour of tissue. To access the treatment site, an opening is formed for insertion of the guide into the patient's body. The ablation segment is brought into contact with the desired ablation site and maintained at a temperature (as measured internally of the segment) ranging from about 100 degrees Celsius to about −200 degrees Celsius, while resting in contact with the tissue site for a period varying from several seconds to several minutes, e.g., about one to two minutes. The temperature as measured inside the tip may be correlated with a somewhat higher tissue interface contact temperature by empirical calibration measurements if desired in order to implement various treatment control regimens. The described system could include a contact assessment system, which allows the assessment of good contact across the cooling segment and the tissue structure. The contact assessment system could include an impedance measurement or impedance scanning between sets of electrodes placed on the cooling segment or a temperature change measurement system. The ablation segment contains a series of thermocouples placed at identified location. The temperature gradient between the two or more thermocouples provided information about the surface contact between the ablation segment and the tissue. Alternatively, the temperature from all the sensors can be monitored to provide information about the surface contact between the ablation segment and the tissue.

All patents, patent applications and publications referred to or cited herein, or from which a claim for benefit of priority has been made, are incorporated by reference in their entirety to the extent they are not inconsistent with the explicit teachings of this specification, including: U.S. Pat. No. 6,270,476 to Santoianni et al.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 illustrates the medical device of the subject invention;

FIG. 2 is a sectional view of the medical device of the subject invention;

FIG. 3 is front sectional view Of the distal end of the medical device of the subject invention including an insulating pad;

FIG. 4 is a section view of the distal end of the medical device of the subject invention;

FIG. 5 illustrates the slotted segment at the distal end of the shapeable guide of the subject invention;

FIG. 6 is a section view of the distal end of the medical device of the subject invention including an insulating pad;

FIG. 7 is a section view of the distal end of the medical device of the subject invention including slideably tracks in the guide;

FIG. 8 is a sectional view of a cryogenic ablation tool of the subject invention;

FIG. 9 is a sectional view of a cryogenic ablation tool of the subject invention including fluid outlets in the ablation segment; and

FIG. 10 is a schematic illustration of an embodiment of a cryosurgical system in accordance with the invention

### DETAILED DESCRIPTION OF THE INVENTION

The present invention provides a medical device and ablation tool for guiding an ablation tool onto the surface of tissue. As described herein, the invention includes a device which can be shaped to reach around an organ, bone or tissue structure, and have an optimal configuration for positioning and/or orienting the active or distal region of the device based upon the particular anatomy of a patient and the location of the treatment site.

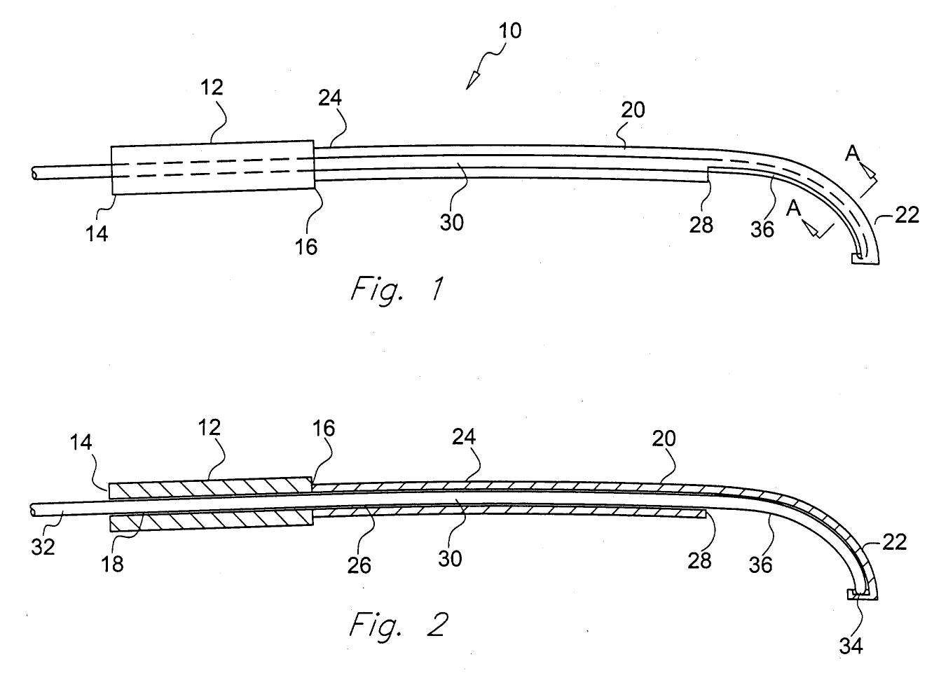

Referring to FIG. 1, an exemplary medical device 10 includes a sheath handle 12 having a proximal portion 14 and a distal portion 16, and in this embodiment includes a shapeable guide 20 extending from the handle distal portion 16 for positioning a distal end 22 of the guide 20. A flexible ablation tool 30 is positioned within the sheath handle 12 and shapeable guide 20, such that an ablation segment 36 is located within the distal end 22 of the guide 20. As described below, in use the shapeable guide 20 is bent by a surgeon or other medical personnel into a conformation, particular for the anatomy of a patient or a desired legion shape, to access the location of the tissue to be treated and position the ablation segment 36 in an orientation to treat the tissue.

As shown in FIG. 2, the sheath handle 12 has a central lumen 18 formed therein to accommodate the ablation tool 30 that ultimately extends to the distal end 22 of the guide. Generally, the ablation tool 30 extends proximally from the proximal portion 14 of the sheath handle 12 for connection to a remote control device.

The guide 20 has a proximal end 24 and distal end 22, and defines a central lumen 26, where the proximal end 24 of the guide 20 is attached to and extending from the distal portion 16 of the sheath handle 12. The distal end 22 of the guide 20 further includes a slotted segment 28, such that a portion of the central lumen 26 is exposed.

The flexible ablation tool 30, having a proximal end 32 and distal end 34, is positioned through and within the handle lumen 18 and guide lumen 26, such that the distal end 34 of the ablation tool 30 is in proximal relation with the distal end 22 of the guide 20. The distal end 34 of the ablation tool 30 further includes an ablation segment 36, which is positioned in the slotted segment 28 of the guide 20, such that a portion of the ablation segment 36 is exposed to the tissue to be treated.

Additionally, the guide 20 has a shape-holding deformability, that is, it has a rigidity such that the guide 20 retains a first shape until manipulated to a further shape with the application of moderate pressure, and until reshaped. The guide 20 retains its shape with sufficient rigidity to manipulate the distal end 22 of the guide 20, urging the ablation segment 36 against tissue, and push it past intervening tissue to a desired position. It is understood that shape, as used herein, is to be construed broadly to include any contour which is needed to configure the medical device 10 for reaching an obscure or distal location in the body for positioning the active or distal portion of the ablation tool 30, and may include successive bends or segments having more than one curve, angle, deformation or other non-linear configuration. The shape-retaining feature of the guide 20 allows an operator to bend the guide 20 to a shape or contour, for example to reach around an organ, bone or tissue structure, and have an optimal configuration for positioning and or orienting the active or distal region of the medical device 10 based upon the particular anatomy of a patient and the location of the treatment site. Further, the stiffness of the guide 20 is such that the surgeon can form the guide 20 by hand to a desired shape without undue effort, and yet the guide 20 retains the set shape as the medical device 10 is maneuvered to and held in position at the treatment site. The guide 20 should also be sufficiently rigid such that the surgeon can place the ablation segment 36 of the ablation tool 30 in pressured contact with the tissue treatment site. That is, the guide 20 is sufficiently stiff to enable the surgeon to press the ablation segment 36 against the tissue to be treated without inducing a further deformation in the shape of the guide 20. The guide 20 may in some embodiments deflect slightly, and yet has sufficient stiffness to transfer an effective level of lateral force at its distal end.

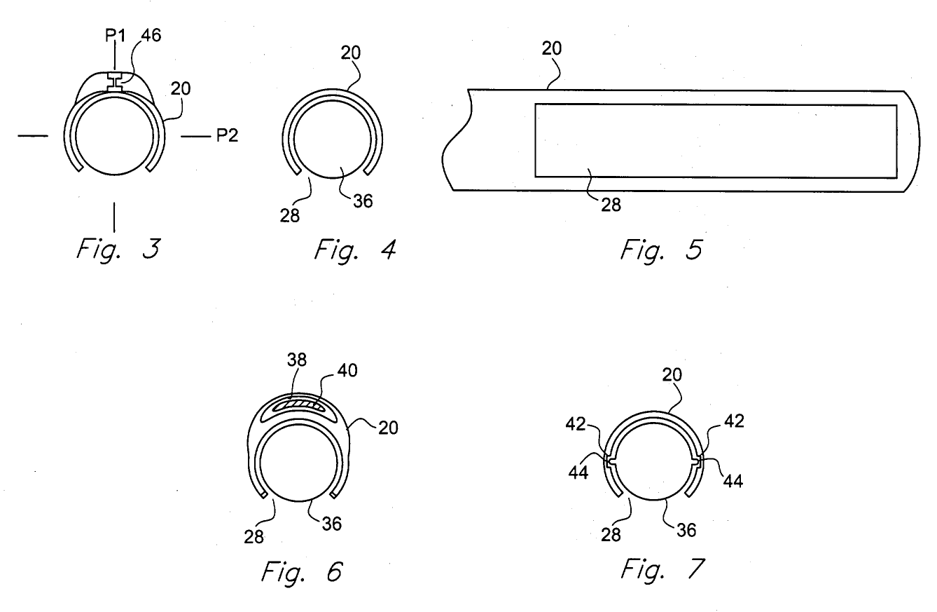

Referring to FIG. 3, the guide is configured so that it is deformable in a single plane, where the guide remains substantially rigid in all other planes. For example, the guide includes an elongated member 46 which can be manipulated in a first plane “P1” from a first shape to a second shape, wherein the elongated member 46 is sufficiently rigid to retain the second shape. The elongated member 46 also has sufficient rigidity such that the guide 20 cannot be manipulated in a second plane “P2” orthogonal to the first plane, such that the guide is deformable only in the first plane. As such the guide 20 is deformable in only one plane.

In accordance with yet another aspect of the invention, as shown in FIGS. 4-5, particularly directed to the ablative properties of ablation segment 36 of the ablation tool 30, the energy distribution during treatment of tissue is further controlled by the slotted segment 28, which acts like a insulating sheath extending over a partial circumference of the ablation segment 36. In this embodiment, the slotted segment 28 forms a partial circumferential blanket or insulating pad which prevents the ablation segment 36 from affecting tissue on one side of the guide distal end 22, while leaving the other side of the ablation segment 36 exposed for contact with tissue. The depiction of the guide having a circular cross section is only exemplary, and the guide may have non-circular cross sections, including, but not limited to, elliptical, rectangular, or triangular.

In accordance with yet another aspect of the invention, as shown in FIG. 6, the guide 20 further includes an upper lumen 38 interposed between the ablation tool 36 and the adjacent tissue, the upper lumen 38 being configured to receive an insulating pad 40, the insulating pad 40 partially circumferencing the ablation tool 30 and acting to provide shielding to prevent undesirable damage to the adjacent tissue. The insulating pad 40 can also be made of a material that affects the malleability of the guide 20.

In a further embodiment, as shown in FIG. 7, the guide 20 includes longitudinal tracks 42 within the central lumen 26 surface. The tracks 42 are configured for receiving a pair of rail 44 disposed on opposing side of the ablation tool 30, such that the rails are slidable with the tracks 44 and the ablation tool 30 is secured with the guide 20.

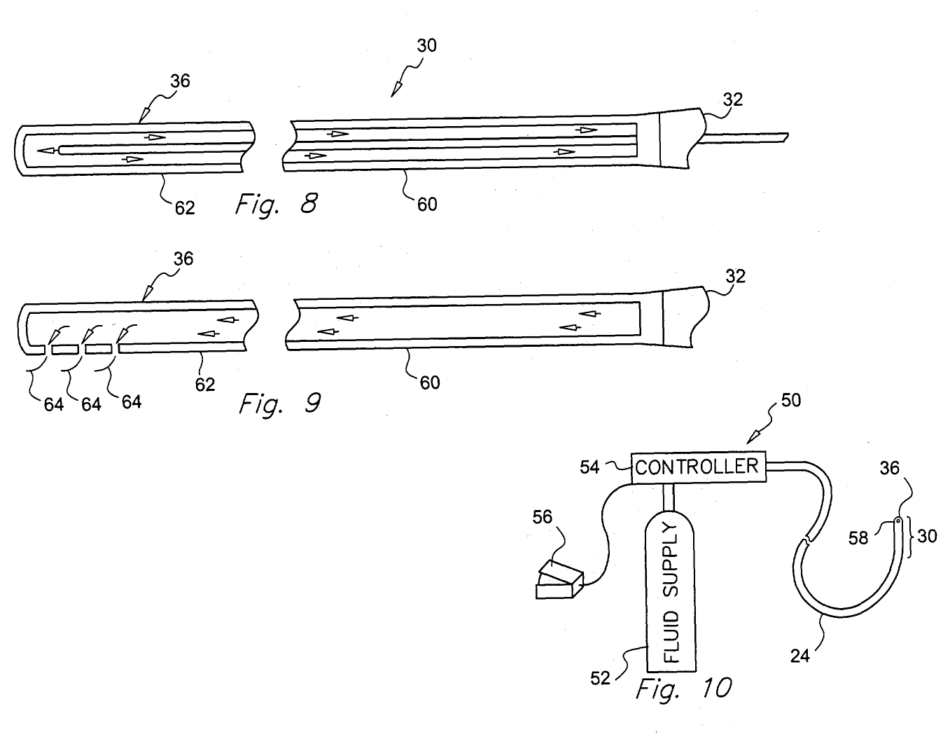

In an exemplary embodiment, as shown in FIG. 8, the ablation tool 30 includes a flexible member 60 having an ablation segment 36 having a thermally-transmissive region 62, and a fluid path through the flexible member to the ablation segment 36. A fluid path is also provided from ablation segment 36 to a point external to the ablation tool 30, such as the proximal end 32. An exemplary fluid path can be one or more channels defined by the flexible member 60, and/or by one or more additional flexible members that are internal to the first flexible member 60. Also, even though many materials and structures can be thermally conductive or thermally transmissive if chilled to a very low temperature and/or cold soaked, as used herein, a “thermally-transmissive region” is intended to broadly encompass any structure or region of the ablation tool 30 that readily conducts heat.

For example, a metal structure exposed (directly or indirectly) to the cryogenic fluid path is considered a thermally-transmissive region 62 even if an adjacent polymeric or latex catheter portion also permits heat transfer, but to a much lesser extent than the metal. Thus, the thermally-transmissive region 62 can be viewed as a relative term to compare the heat transfer characteristics of different catheter regions or structures, regardless of the material.

Furthermore, while the thermally-transmissive region 62 can include a single, continuous, and uninterrupted surface or structure, it can also include multiple, discrete, thermally-transmissive structures that collectively define a thermally-transmissive region that is elongate or linear. Depending on the ability of the cryogenic system, or portions thereof, to handle given thermal loads, the ablation of an elongate tissue path can be performed in a single or multiple cycle process without having to relocate the catheter one or more times or drag it across tissue.

In an embodiment, as shown in FIG. 9, the ablation segment 36 includes one or more orifices 64, where the orifices 64 defines the thermally-transmissive region 62. The orifices 64 enable the application of cryogenic fluid directly onto the tissue to be treated.

In an exemplary embodiment, as shown in FIG. 10, the present invention includes cryosurgical system 50 in accordance with the invention. The system includes a supply of cryogenic or cooling fluid 52 in communication with the proximal end 32 of a flexible ablation tool 30. A fluid controller 54 is interposed or in-line between the cryogenic fluid supply 55 and the ablation tool 30 for regulating the flow of cryogenic fluid into the ablation tool 30 in response to a controller command. Controller commands can include programmed instructions, sensor signals, and manual user input. For example, the fluid controller 54 can be programmed or configured to increase and decrease the pressure of the fluid 52 by predetermined pressure increments over predetermined time intervals. In another exemplary embodiment, the fluid controller 54 can be responsive to input from a user input device 56 to permit flow of the cryogenic fluid into the ablation tool 30. One or more temperature sensors 58 in electrical communication with the controller 54 can be provided to regulate or terminate the flow of cryogenic fluid into the ablation tool 30 when a predetermined temperature at a selected point or points on or within the ablation segment 36 is/are obtained. For example a temperature sensor 58 can be placed at a point proximate the ablation tool distal end 34 and other temperature sensors 58 can be placed at spaced intervals between the ablation tool distal end 34 and another point that is between the distal end 34 and the proximal end 32.

In another exemplary embodiment, the fluid controller 54 can be responsive to input from a user input device 56 to permit flow of the cryogenic fluid into the ablation tool 30. One or more sensors, such as a ECG leads, in electrical communication with the controller 54 can be provided to regulate or terminate the flow of cryogenic fluid into the ablation tool 30 depending on the electrical activity in the tissue being treated. For example an electrical sensor can be placed at a point proximate the ablation tool distal end 34 and other electrical sensor can be placed at spaced intervals between the ablation tool distal end 34 and another point that is between the distal end 34 and the proximal end 32.

Alternatively, the electrical sensors can be pressure sensors. The pressure sensors can be used to determine when the ablation segment 36 is in physical contact with the tissue to be treated.

The cryogenic fluid can be in a liquid or a gas state. An extremely low temperature can be achieved within the medical device 10, and more particularly at the ablation segment 36 by cooling the fluid to a predetermined temperature prior to its introduction into the medical device 10, by allowing a liquid state cryogenic fluid to boil or vaporize, or by allowing a gas state cryogenic fluid to expand. Exemplary liquids include chlorodifluoromethane, polydimethylsiloxane, ethyl alcohol, HFC's such as AZ-20 (a 50—50 mixture of difluoromethane & pentafluoroethane sold by Allied Signal), and CFC's such as DuPont's Freon. Exemplary gasses include argon, nitrous oxide, and carbon dioxide.

Although generally shown as a cryogenic ablation tool, it is understood that in other embodiments the ablation segment 36 applies other types of energy or combination of energies, to the tissue to be treated, including, but not limited to, cryogenic energy, radio frequency (RF) energy, microwave energy, ultrasound energy, laser energy, and contact heating energy. It is further understood that other devices can be coupled to the guide distal end 22, for example, cameras, video devices, probes and other components can be affixed to the guide 20 for various applications For example, pacing/sensing electrodes can be affixed to points on ton the slotted segment 28.

The medical device 10 of the present invention is well suited for treating tissue in a variety of locations in the body during invasive surgical procedures. Illustrative applications include open thoracic and peritoneal surgery as well as endoscopic procedures, e.g., treating tissue located at or near the heart, intestines, uterus, and other regions for which surgical or endoscope assisted surgical access and topical tissue treatment, or cauterization or ablation is appropriate, as well as ophthalmic surgery, and tumor ablation and various applications preparatory to further surgical steps.

In an illustrative application, the medical device 10 is used to treat cardiac arrhythmias. The patient will in general be examined, for example with known cardiac mapping, fluoroscopy, endoscopic camera, and other soft tissue imaging techniques, or such techniques in conjunction with a mapping catheter with mapping electrodes, so as to determine accurate anatomic heart characteristics and signal pathways, and to identify and map the location of tissue to be treated. Based on the patient's anatomy and treatment site, the guide 20 is shaped to achieve an optimal configuration for reaching and orienting the ablation segment in physical contact with the target tissue for ablating a line or contour.

To access the treatment site, an opening is formed for insertion of the medical device 10 into the patient's body. For example, to ablate a linear ablation line on the wall of the atrium, a chest opening provides access to the heart. The medical device 10 may be inserted into the atrium via a local cut to form, for example, an elongated lesion on the atrial wall. Most preferably, however, the medical device 10 of the present invention is used to form epicardial ablation lines, for example to reach around to the posterior outer surface of the heart and form ablation lines in an occluded region. In an illustrative treatment, the ablation segment 36 is brought into contact with the desired ablation site and maintained at a temperature (as measured internally of the segment) ranging from about 37 degrees Celsius to about −200 degrees Celsius, while resting in contact with the tissue site for a period of several minutes, e.g., about five minutes. The temperature as measured inside the tip may be correlated with a somewhat higher tissue interface contact temperature by empirical calibration measurements if desired in order to implement various treatment control regimens.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A medical device for ablating tissue comprising a guide defining a central lumen, the guide having a malleable conformation such that the guide retains a first shape until manipulated to a second shape.

2. The medical device according to claim 1, further comprising an ablation tool including an ablation segment, wherein the ablation tool is disposed with the central lumen.

3. The medical device according to claim 2, wherein the guide is configured for receiving the ablation tool, such that at least a portion of the ablation segment is exposed to the tissue to be treated.

4. The medical device according to claim 3, wherein the guide is configured to partially surround the exposed portion of the ablation segment.

5. The medical device according to claim 4, wherein the guide further includes an insulation pad configured to partially surround the ablation tool.

6. The medical device according to claim 2, wherein the guide further includes a track configured for slideably receiving the ablation tool.

7. The medical device according to claim 2, wherein the ablation tool is operably connected to an ablation control system.

8. The medical device according to claim 7, further including at least one temperature sensor positioned in thermal relation to the ablation segment and operably connected to the ablation control system.

9. The medical device according to claim 7, further including at least one signal sensor positioned in proximal relation to the ablation segment and operably connected to the ablation control system.

10. The medical device according to claim 2, wherein the ablation tool is configured to circulate cryogenic fluid therethrough for ablation of the tissue contacting the ablation tool.

11. The medical device according to claim 10, wherein the ablation tool includes at least one orifice such that cryogenic fluid can be applied directly to the tissue.

12. The medical device according to claim 2, wherein the ablation tool is configured to transfer ablation energy selected from the group consisting of cryogenic energy, radio frequency energy, microwave energy, ultrasound energy, laser energy, chemical energy, and contact heating energy.

13. The medical instrument according to claim 12, wherein the ablation tool is configured to transfer a combination of ablation energy.

14. A medical instrument comprising:
a handle;
a shapeable guide extending distally from the handle defining a central lumen therethrough, the shapeable guide including a malleable conformation such that the shapeable guide retains a first shape until manipulated to a second shape, wherein the second shape conforms to the particular anatomy of a patient;
a flexible ablation tool including an ablation segment, wherein the ablation tool is disposed within the central lumen.

15. The medical instrument according to claim 14, wherein the shapeable guide is malleable within a first plane and is substantially rigid within a second plane orthogonal to the first plane.

16. The medical instrument according to claim 14, wherein the shapeable guide includes a slotted segment substantially located at a guide distal end, such that the ablation segment is substantially positioned within the slotted segment.

17. The medical instrument according to claim 16, wherein the slotted segment is configured to expose at least a portion of the ablation segment, such that the shapeable guide acts as an insulator preventing undesirable damage to tissue adjacent to the unexposed portion of the ablation segment.

18. The medical device according to claim 14, wherein the shapeable guide further includes an insulation pad configured to partially surround the ablation tool.

19. The medical device according to claim 14, wherein the shapeable guide further includes a track configured for slideably receiving the ablation tool.

20. The medical instrument according to claim 14, wherein the ablation tool is configured to circulate cryogenic fluid therethrough for ablation of tissue contacting the ablation segment.

21. The medical instrument according to claim 20, further including a cryogenic fluid control system, including a controller operable connected to a cryogenic fluid supply, wherein the fluid supply is connected to the ablation tool.

22. The medical instrument according to claim 20, furthering including at least one temperature sensor positioned in thermal relation to the ablation segment and operable connected to the controller.

23. The medical instrument according to claim 20, furthering including at least one electrical signal sensor positioned in proximal relation to the ablation segment and operable connected to the controller.

24. The medical instrument according to claim 14, wherein the ablation tool is configured to transfer ablation energy selected from the group consisting of cryogenic energy, radio frequency energy, microwave energy, ultrasound energy, laser energy, chemical energy, and contact heating energy.

25. The medical instrument according to claim 24, wherein the ablation tool is configured to transfer a combination of ablation energy.

26. A medical instrument for ablation of tissue comprising:
a handle including a handle proximal end, a handle distal, and a handle lumen defining a path therethrough;
a shapeable guide including a guide proximal end, a guide distal end, a slotted segment, and a guide lumen defining a path there through, wherein the slotted segment is substantially located at the guide distal end such that a portion of the guide lumen is exposed, the handle distal end and the guide proximal end being affixed such that the handle lumen and the guide lumen define a path there through; and
a flexible ablation tool including an ablation tool proximal end, an ablation tool distal end, and an ablation segment, the ablation segment being substantially located at the ablation tool distal end, wherein the flexible ablation tool is inserted through the handle lumen and the guide lumen, such that the ablation segment is position substantially with the slotted segment and the ablation tool proximal end extends from the handle proximal end.

27. The medical device according to claim 26, further comprising a cryogenic fluid source connected to the ablation tool distal end.
